(19) United States
(12) Patent Application Publication
(10) Pub. No.: US 2016/0199825 A1
(43) Pub. Date: Jul. 14, 2016

(54) COMPOSITE IONIC LIQUID CATALYST

(71) Applicants: SHELL OIL COMPANY, Houston, TX (US); CHINA UNIVERSITY OF PETROLEUM, Beijing (CN)

(72) Inventors: Rui ZHANG, Beijing (CN); Jan DE WITH, Amsterdam (NL); Peter Anton August KLUSENER, Amsterdam (NL); Zhichang LIU, Beijing (CN)

(21) Appl. No.: 14/914,541

(22) PCT Filed: Aug. 27, 2014
(86) PCT No.: PCT/EP2014/068179

§ 371 Date: Feb. 25, 2016

(30) Foreign Application Priority Data
Aug. 29, 2013 (CN) PCT/CN2013/082516

(51) Int. Cl. B01J 31/02; C07C 2/58

(52) U.S. Cl. CPC B01J 31/0284; B01J 31/0279; C07C 2/58; B01J 2231/44; C07C 2531/02

(57) ABSTRACT

The present invention relates to a composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the ammonium cation is a N,N′-disubstituted imidazolium cation, the substituents independently being selected from C1-C10 alkyl, and C6-C10 aryl. The composite ionic liquid of the invention is a stable catalyst, which can suitably be used to run an ionic liquid alkylation process which produces less solids and an alkylate product comprising less organic chlorides as side products than processes known from the prior art.

### FIELD OF THE INVENTION

The present invention provides a new composite ionic liquid catalyst, a process for preparing an alkylate using the new catalyst and a process for the preparation of a composite ionic liquid catalyst.

### BACKGROUND OF THE INVENTION

There is an increasing demand for alkylate fuel blending feedstock. As a fuel-blending component alkylate combines a low vapour pressure, no sulfur, olefins or aromatics with high octane properties. The most desirable components in the alkylate are trimethylpentanes (TMPs), which have research octane numbers (RONs) of greater than 100. Such an alkylate component may be produced by reacting isobutane with a butene in the presence of a suitable acidic catalyst, e.g. HF or sulfuric acid, although other catalysts such a solid acid catalyst have been reported. Recently, the alkylation of isoparaffins with olefins using an ionic liquid catalyst has been proposed as an alternative to HF and sulfuric acid catalysed alkylation processes.

For instance, U.S. Pat. No. 7,285,698 discloses a process for manufacturing an alkylate oil, which uses a composite ionic liquid catalyst to react isobutane with a butene. Said composite ionic catalyst comprises ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table. Although that composite ionic liquid catalyst can suitably be used in alkylation processes as an alternative to HF and sulfuric acid catalysed alkylation processes, the use of the composite ionic liquid catalyst is accompanied with some drawbacks: solids formation in the reaction system during use and the production of organic chlorides as side products. Solids formation means unwanted catalyst consumption and potential risks of blocking the pipelines in the reaction system. Further, the presence of organic chlorides in the product undermines the quality of the alkylate and will corrode the engine when used in a fuel. The organic chlorides will either need to be removed from the product stream or the content of organic chlorides in the product stream will need to be reduced otherwise.

### SUMMARY OF THE INVENTION

A new composite ionic liquid catalyst has now been found, the use of which leads to reduction of organic chlorides in the alkylate product. In addition, less solids may form while using this new catalyst when compared to composite ionic catalysts known in the art. Further, the new catalyst shows selectivity towards the production of trimethylpentanes. In addition, composite ionic liquids of the present invention show improved stability; the lifetime is longer than composite ionic catalysts known in the art.

Accordingly, the present invention provides a composite ionic liquid catalyst comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the ammonium cation is a N,N′-disubstituted imidazolium cation, the substituents independently being selected from C1-C10 alkyl, and C6-C10 aryl.

### DETAILED DESCRIPTION OF THE INVENTION

The presently claimed catalyst is a composite ionic liquid comprising ammonium cations being N,N′-disubstituted imidazolium cations, optionally further substituted at the 2-, 4- and/or 5-positions, wherein the substituents independently are selected from C1-C10 alkyl, and C6-C10 aryl.

In particular, those substitutents are selected from C1-C6 alkyl and phenyl. Preferably, the ammonium cation is a N-butyl, N′-methylimidazolium, optionally substituted with methyl at the 2-position. It was found that N-butyl, N′-methylimidazolium composite ionic liquid performed better under alkylation conditions than ionic liquid known in the prior, which includes improved alkylate distribution, lower organic chlorides content, less solids amount and improved lifetime. Most preferably, the imidazolium cation is N-t-butyl, N′-methylimidazolium. A preferred composite ionic liquid is [t-Bmim]C1-1.8AlCl₃-0.5CuCl, which reduces the production of organic chloride as side products. Furthermore, when used for alkylation, the [t-Bmim]C1-1.8AlCl₃-0.5CuCl composite ionic liquid catalyst results in high selectivity for C₈ alkylate production.

The anions of the composite ionic liquid are derived from aluminium based Lewis acids, in particular aluminium halides, preferably aluminium (III) chloride. Due to the high acidity of the aluminium Lewis acid the aluminium chloride, or other aluminium halide, is combined with a second or more metal halide, sulfate or nitrate, to form a coordinate anion, in particular a coordinate anion derived from two or more metal halides, wherein at least one metal halide is an aluminium halide. Suitable further metal halides, sulfates or nitrates, may be selected from halides, sulfates or nitrates of metals selected from the group consisting of Group IB elements of the Periodic Table, Group IIB elements of the Periodic Table and transition elements of the Periodic Table. Preferred metals include copper, iron, zinc, nickel, cobalt, molybdenum, silver or platinum, in particular copper. Preferably, the metal halides, sulfates or nitrates, are metal halides, more preferably chlorides or bromides, most preferably copper (I) chloride. Particularly preferred catalysts are acidic ionic liquid catalysts comprising a coordinate anion derived from aluminium(III) chloride and copper(I) chloride.

In an embodiment of the invention, the molar ratio of the aluminium salt to the ammonium salt ranges from 1.2 to 2.2, preferably 1.6 to 2.0, and more preferred 1.7 to 1.9, and most preferably the ratio is 1.8.

Preferably, the molar ratio of the aluminium salt to the other metal salt(s) in the range of from 1:100-100:1, more preferably of from 1:1-100:1, or even more preferably of from 2:1-30:1, and in particular the range is from 2.5:1-5:1. In an embodiment of the invention, the ratio of AlCl₃ to CuCl is 3.6:1.

In a further embodiment, the molar ratio of the further metal salt(s), in particular the copper salt, to the ammonium salt ranges from 0.3 to 0.7, preferably 0.4 to 0.6, most preferably the ratio is 0.5.

Another embodiment of the invention relates to a process for the preparation of a composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, in which process the two or more metal salts are (first) mixed, for instance portion-wise, with the ammonium cations, in the form of an ammonium salt, and (subsequently) the mixture is kept at a temperature of 120 to 170° C. while stirring until all solids have completely converted into the liquid phase. “Portion-wise” as referred herein means “in at least two portions”. Accordingly, in a portion-wise addition mode, at least (a total of) two portions of the two or more metal salts (e.g. AlCl₃ and CuCl) are added in at least (a total of) two steps to the ammonium salt and mixed with each other. The reaction of the metal salts with the ammonium salt is fast and exothermic. The size of the portions of the metal salts is selected such that the temperature raise is controlled. The mixing time between the addition of the first portion of metal salt and the addition of a subsequent portion is dependent on the nature of the exothermic effect of the addition of the metal salt. The temperature after addition and mixing of a portion of a metal salt into the ammonium salt or ammonium salt mixture, the latter comprising the ammonium salt and one or more portions of the two or more metal salts, should preferably be kept such that the reactor pressure is higher than the vapour pressure of the aluminium salt at the given temperature. Thus at atmospheric pressure and using aluminium chloride as the aluminium salt the temperature should be kept below 180° C. and preferably below 160° C. to avoid loss of aluminium chloride. It is noted here that the mixing of the two or more metal salts in this process is not limited to the portion-wise addition mode. Any method to add the metal salts in a manner that controls the heat production may be suitable. Thus, any technical options known in the art for controlled continuous dosing of solids may be applied.

It is preferred in the process of preparation of the composite ionic liquid to keep the reaction mixture at 120 to 160° C. for an extended period of time, preferably at least 4 hours, more preferred for at least 8 hours, up to about 12 hours, after the addition of the aluminium salt, preferably aluminium chloride.

Preferably, in the process of the invention, the composite ionic liquid is a composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the ammonium cation is a N,N′-disubstituted imidazolium cation, the substituents independently being selected from C1-C10 alkyl, and C6-C10 aryl.

As mentioned herein above, the composite ionic liquid of the invention is used for the production of alkylate. Thus, another embodiment of the invention relates to a process for preparing an alkylate comprising contacting in a reaction zone a hydrocarbon mixture, comprising at least an isoparaffin and an olefin, with a composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the ammonium cation is a N,N′-disubstituted imidazolium cation, the substituents independently being selected from C1-C10 alkyl, and C6-C10 aryl.

Accordingly, the hydrocarbon mixture is mixed in the reaction zone with the catalyst to form a reaction mixture to react under alkylation conditions. Mixing of the hydrocarbon mixture and the catalyst may be done by any suitable means for mixing two or more liquids, including dynamic and static mixers. As the reaction progresses, the reaction mixture will comprise alkylate products in addition to the hydrocarbon reactants (isoparaffins and olefins) and the composite ionic liquid catalyst.

The formed alkylate is obtained from the reaction zone in the form of an alkylate-comprising effluent. The alkylate-comprising effluent still comprises a substantial amount of unreacted isoparaffin. Preferably a part of the alkylate-comprising effluent is recycled to the reaction zone in order to maintain a high ratio of isoparaffin to olefin in hydrocarbon mixture in the reaction zone.

Further, at least part of the alkylate-comprising effluent from the reaction zone is separated in a separator unit into a hydrocarbon-rich phase and an ionic liquid catalyst-rich phase. At least part of the hydrocarbon-rich phase is treated and/or fractionated (e.g. by distillation) to retrieve the alkylate and optionally other components present in the hydrocarbon-rich phase, such as unreacted isoparaffin or n-paraffins. Preferably, such isoparaffin is at least partly reused to form part of the isoparaffin feed provided to the process. This may be done by recycling at least part of the isoparaffin, or a stream comprising isoparaffin obtained from the fractionation of the hydrocarbon-rich phase, and combining it with the isoparaffin feed to the process.

Reference herein to a hydrocarbon-rich phase is to a phase comprising more than 50 mol % of hydrocarbons, based on the total moles of hydrocarbon and ionic liquid catalyst.

Reference herein to an ionic liquid catalyst-rich phase is to a phase comprising more than 50 mol % of ionic liquid catalyst, based on the total moles of hydrocarbon and ionic liquid catalyst.

Due to the low affinity of the ionic liquid for hydrocarbons and the difference in density between the hydrocarbons and the ionic liquid catalyst, the separation between the two phases is suitably done using for example well known settler means, wherein the hydrocarbons and catalyst separate into an upper predominantly hydrocarbon phase and lower predominantly catalyst phase or by using any other suitable liquid/liquid separator. Such liquid/liquid separators are known to the skilled person and include cyclone and centrifugal separators. The catalyst phase is generally recycled back to the reactor.

As described herein before, during the alkylation reaction some solids are formed in the reaction zone. Reference herein to solids is to non-dissolved solid particles. The solids predominantly consist out of metals, metal compounds and/or metal salts which were originally comprised in the composite ionic liquid catalyst. The solids may comprise at least 10 wt % metal, i.e. either in metallic, covalently bound or ionic form, based the total weight of the solids, wherein the metal is a metal that was introduced to the process as part of the ionic liquid catalyst. The solids may also comprise contaminant components, which were introduced into the reaction mixture as contaminants in the hydrocarbon mixture or the composite ionic liquid. Alternatively, the solids may be the product of a chemical reaction involving any of the above-mentioned compounds.

A high solids content in the reaction zone may result in blockage of pathways or valves in the reactor zone and pipes to and from the separation unit, due to precipitation of solids. In addition, at high solids content the solids may agglomerate to form large aggregates, resulting in increased blockage risk. Therefore, preferably at least part of the solids is removed from the reaction zone. It is not required to remove all solids from the reaction zone. Preferably, solids are removed from the reaction zone to an extent that the reaction mixture (i.e. a mixture comprising hydrocarbon reactants, composite ionic liquid and products) comprises in the range of from 0.05 to 5 wt %, more preferably at most 2 wt % of solids, based on the total weight composite ionic liquid in the reaction zone.

The solids may be removed from the reaction zone at any time or place in the process and by any suitable means for removing solids from liquids. It is possible to remove the solids from the reaction mixture directly inside the reaction zone. However, preferably, at least part of the reaction mixture is withdrawn from the reaction zone as a solids-comprising effluent. This solids-comprising effluent comprises next to the solid also hydrocarbons and composite ionic liquid, wherein the hydrocarbons typically include isoparaffins and alkylate. Subsequently, preferably at least part of the solids in at least part of the solids-comprising effluent is removed. After the removal of solids a solids-depleted effluent is obtained. Preferably, at least part of the solids-depleted effluent is recycled to the reactor for efficient use of the materials.

Some further process details of the alkylation process are described here. Preferably, the hydrocarbon mixture comprises at least isobutane and optionally isopentane, or a mixture thereof, as an isoparaffin. The hydrocarbon mixture preferably comprises at least an olefin comprising in the range of from 2 to 8 carbon atoms, more preferably of from 3 to 6 carbon atoms, even more preferably 4 or 5 carbon atoms. Examples of suitable olefins include, propene, 1-butene, 2-butene, isobutene, 1-pentene, 2-pentene, 2-methyl-1-butene, 3-methyl-1-butene, 2-methyl-2-butene.

Isoparaffins and olefins are supplied to the process in a molar ratio, which is preferably 1 or higher, and typically in the range of from 1:1 to 40:1, more preferably 1:1 to 20:1. In the case of a continuous process, excess isoparaffin can be recycled for reuse in the hydrocarbon mixture.

The temperature in the alkylation reactor is preferably in the range of from −20 to 100° C., more preferably in the range of from 0 to 50° C. In any case the temperature must be high enough to ensure that the ionic liquid catalyst is in the liquid state.

To suppress vapour formation in the reactor, the process may be performed under pressure; preferably the pressure in the reactor is in the range of from 0.1 to 1.6 MPa.

Preferably, the ratio of composite ionic liquid catalyst to hydrocarbon in the alkylation reaction zone is at least 0.5, preferably 0.9, more preferably at least 1. Preferably, the ratio of composite ionic liquid catalyst to hydrocarbon in the reaction zone is in the range of from 1 to 10.

The hydrocarbon mixture may be contacted with the catalyst in any suitable alkylation reactor. The hydrocarbon mixture may be contacted with the catalyst in a batch-wise, a semi-continuous or continuous process. Reactors such as used in liquid acid catalysed alkylation can be used (see L. F. Albright, Ind. Eng. Res. 48 (2009)1409 and A. Corma and A. Martinez, Catal. Rev. 35 (1993) 483); alternatively the reactor may be a loop reactor, optionally with multiple injection points for the hydrocarbon feed, optionally equipped with static mixers to ensure good contact between the hydrocarbon mixture and catalyst, optionally with cooling in between the injection points, optionally by applying cooling via partial vaporization of volatile hydrocarbon components (see Catal. Rev. 35 (1993) 483), optionally with an outlet outside the reaction zone (see WO2011/015636). In several publications diagrams are available of alkylation process line-ups which are suitable for application in the process of this invention, e.g. in U.S. Pat. No. 7,285,698, Oil & Gas J., vol 104 (40) (2006) p 52-56 and Catal. Rev. 35 (1993) 483.

### LEGENDS TO THE DRAWINGS

FIG. 1. TMP content of alkylate, catalysis by [Bmim]Cl-xAlCl₃-0.5CuCl

FIG. 2. DMH (dimethylhexane) content of alkylate, catalysis by [Bmim]Cl-xAlCl₃-0.5CuCl

FIG. 3. RON and Cl content of alkylate, catalysis by [Bmim]Cl-xAlCl₃-0.5CuCl

The invention is illustrated by the following non-limiting examples.

### Example 1

### Preparation of Et₃NHCl Composite IL (IL-1)

Et₃NHCl (1 mol) was placed in a 500 mL flask under N₂ atmosphere. Subsequently, AlCl₃ (0.45 mol) was added into the flask. A reaction started and the solids liquefied. The mixture was stirred while the temperature raised to 100° C. by the exothermic reaction. When the temperature had decreased below 60° C. by cooling to the atmosphere another portion of AlCl₃ (0.45 mol) was added to the IL mixture. The temperature of IL rose to 120° C. while cooling to atmosphere. Then CuCl (0.5 mol) was added to the IL mixture. The IL mixture was heated as soon as the temperature started to drop and kept at 120° C. for at least 2 hours by external heating. Then a third portion of AlCl₃ (0.45 mol) was added into the flask. The temperature of IL rose to 150° C. The last portion of AlCl₃ (0.45 mol) was added into the flask as soon as the temperature started to drop. The temperature of mixture was kept at 150° C. for at least 4 hours using external heating, after which the composite IL was allowed to cool down to room temperature.

### Example 2

### Preparation of BmimCl Composite IL (IL-2)

The procedure of example 1 was repeated using butyl methyl imidazolium chloride (BmimCl) (1 mol) instead of Et₃NHCl.

### Example 3

### Preparation of [Bmim]C1-1.2AlCl₃-0.5CuCl (IL-3)

1 mol of BmimCl was weighed and put into a 500 mL flask under N₂ atmosphere. 133.34 g of AlCl₃ (1.0 mol) was weighed and put into the flask, and stirring was started. The solids then started to form a liquid phase. Stirring was continued while the temperature of the IL increased by reaction to 80° C. 50 g of CuCl (0.5 mol) was added into the flask. The mixture was heated after the temperature started to drop. The temperature of the mixture was kept at 120° C. for at least 2 hours. Subsequently, 26.27 g of AlCl₃ (0.2 mol) was added into the flask. The temperature of the mixture was kept at 160° C. for 4 hours. Stirring was stopped and the mixture was allowed to cool to room temperature.

### Example 4

### Preparation of [Bmim]C1-1.4AlCl₃-0.5CuCl (IL-4)

The procedure of example 3 was repeated except that instead of the second portion of 0.2 mol of AlCl₃ 0.4 mol of AlCl₃ was added.

### Example 5

### Preparation of [Bmim]C1-1.6AlCl₃-0.5CuCl (IL-5)

The procedure of example 3 was repeated except that instead of the second portion of 0.2 mol of AlCl₃ 0.6 mol of AlCl₃ was added.

### Example 6

### Preparation of [Bmim]C1-1.8AlCl₃-0.5CuCl (IL-6)

The procedure of example 3 was repeated except that instead of the second portion of 0.2 mol of AlCl₃ 0.8 mol of AlCl₃ was added.

### Example 7

### Preparation of [Bmim]C1-1.8AlCl₃-0.5CuCl (IL-7)

The procedure of example 3 was repeated except that instead of the second portion of 0.2 mol of AlCl₃ 1.0 mol of AlCl₃ was added.

### Example 8

### Preparation of [t-Bmim]C1-1.8AlCl₃-0.5CuCl (IL-8)

The procedure of example 6 was repeated using tert-butyl methyl imidazolium chloride (t-BmimCl) (1 mol) instead of BmimCl.

### Example 9

### Preparation of [i-Bmim]C1-1.8AlCl₃-0.5CuCl (IL-9)

The procedure of example 6 was repeated using iso-butyl methyl imidazolium chloride (i-BmimCl) (1 mol) instead of BmimCl.

### Example 10

### Preparation of Et₃NHCl composite IL (IL-10)

137.65 g of Et₃NHCl (1.0 mol) was weighed and put into a 500 mL flask under N₂ atmosphere. 133.34 g of AlCl₃ (1.0 mol) was weighed and put into the flask, and stirring was started. The solids then started to form a liquid phase. Stirring was continued while the temperature of the IL increased by reaction to 80° C. 50 g of CuCl (0.5 mol) was added into the flask. The mixture was heated after the temperature started to drop. The temperature of the mixture was kept at 120° C. for at least 2 hours. Subsequently, 106.67 g of AlCl₃ (0.8 mol) was added into the flask. The temperature of the mixture was kept at 160° C. for 4 hours. Stirring was stopped and the mixture was allowed to cool to room temperature. 427 g of IL-10 was obtained.

### Example 11

### Preparation of Et₃NHCl composite IL (IL-11)

Composite IL-11 was prepared analogously to IL-10, with the exception that after addition of the last portion of AlCl₃ the temperature of the mixture was kept at 120° C. for 4 hours. Stirring was stopped and the mixture was allowed to cool to room temperature. 427 g of IL-11 was obtained.

### Example 12

### Preparation of Et₃NHCl composite IL (IL-12)

Composite IL-12 was prepared analogously to IL-10, with the exception that after addition of the last portion of AlCl₃ the temperature of the mixture was kept at 120° C. for 8 hours. Stirring was stopped and the mixture was allowed to cool to room temperature. 427 g of IL-12 was obtained.

### Example 13

### Preparation of Et₃NHCl composite IL (IL-13) (Comparative Example)

Composite IL-13 was prepared analogously to IL-10, with the exception that after addition of the last portion of AlCl₃ the temperature of the mixture was kept at 100° C. for 4 hours. Stirring was stopped and the mixture was allowed to cool to room temperature. 427 g of IL-13 was obtained.

### Example 14

### Preparation of Et₃NHCl composite IL (IL-14) (Comparative Example)

Composite IL-14 was prepared analogously to IL-10, with the exception that after addition of the last portion of AlCl₃ the temperature of the mixture was kept at 100° C. for 8 hours. Stirring was stopped and the mixture was allowed to cool to room temperature. 427 g of IL-14 was obtained.

### Example 15

### Continuous Alkylation Test with IL-1

60 g of composite IL-1 was placed into an autoclave (280 mL). After the gas cap was flushed with nitrogen the autoclave was closed and the stirrer was started (1000 rpm). The autoclave was controlled at 25° C. The C4 feed consisting of 91.13 wt % of isobutane, 1.68 wt % of n-butane, 3.93 wt % of trans-2-butene, 0.20 wt % of 1-butene, 0.14 wt % of isobutene and 1.52 wt % of cis-2-butene was stored in a feed storage tank. The C4 feed, after filtration, was pumped through a dryer, and then entered into the autoclave at a rate of 500 mL/h. The feed rate was controlled by a plunger pump. The pressure in the autoclave was maintained at 0.6 MPa to keep the reactants and product in liquid phase.

During reaction and filling the autoclave, the reaction system was separating into two phases due to the large difference in density of the ionic liquid and the hydrocarbon layer. The upper part of the autoclave contained the hydrocarbon fraction, while the lower part consisted of a mixture of ionic liquid and hydrocarbon. Samples were taken from the upper layer under pressure through a sample connection into a small sample tank.

A sample taken after 4.0 kg of C4 feed was introduced showed that the olefin conversion was 100%.

After 5.5 kg of C4 feed had been introduced the olefin conversion had dropped to less than 15% and the stirrer was stopped. The autoclave contents were separated into two phases. The lower phase, being a mixture of ILs and solids, was centrifuged to obtain the solids.

The average hydrocarbon composition of the samples taken after 4.0 kg of feed is given in Table 1. The solids production is shown in Table 3.

### Example 16

### Continuous Alkylation Test with IL-2

Example 15 was repeated with the difference that IL-2 was used instead of IL-1. A sample taken after 7.0 kg of C4 feed was introduced showed that the olefin conversion was 100%. After 9.3 kg of C4 feed was introduced the olefin conversion dropped to 15% and the stirred was stopped.

The average hydrocarbon composition of the samples taken after 7.0 kg of feed is given in Table 1. The solids production is shown in Table 3.

### Example 17

### Batch-Wise Alkylation Test with IL-3

C4 feed consisting of 0.15 wt % of propene, 94.23 wt % of isobutane, 9.93 wt % of n-butane, 2.54 wt % of trans-2-butene and 2.13 wt % of cis-2-butene was stored in a feed storage tank. 40 mL of C4 feed, after filtration, was pumped through a dryer, and then entered into a feed tank. The amount of feed was controlled by a plunger pump. 40 mL of composite IL-3 was placed into an autoclave (280 mL). After the gas cap was flushed with nitrogen the autoclave was closed and the stirrer was started (1000 rpm). The temperature of the contents of the autoclave and the feed tank were controlled to 15° C. The feed was pushed within a second into the autoclave by high pressure nitrogen, and the timer was started simultaneously. The pressure in the autoclave was maintained at 0.6 MPa to keep the reactants and product in liquid phase. After 20 seconds the stirrer was stopped and the autoclave was cooled. The reaction system separated immediately into two phases due to the large difference of density of the ionic liquid and the hydrocarbon layer. The reaction time (20 s) in this experiment is defined as the time between the start of the instantaneous feeding and the switch off of the stirrer. After the reaction, a sample was taken under pressure through a sample connection into a small sample tank. The composition data of the alkylate product are given in table 4.

### Example 18

### Batch-Wise Alkylation Test with IL-4

Example 17 was repeated with using IL-4 instead of IL-3 The composition data of the alkylate product are given in table 4.

### Example 19

### Batch-Wise Alkylation Test with IL-5

Example 17 was repeated with using IL-5 instead of IL-3 The composition data of the alkylate product are given in table 4.

### Example 20

### Batch-Wise Alkylation Test with IL-6

Example 17 was repeated with using IL-6 instead of IL-3 The composition data of the alkylate product are given in tables 4 and 5.

### Example 21

### Batch-Wise Alkylation Test with IL-7

Example 17 was repeated with using IL-7 instead of IL-3 The composition data of the alkylate product are given in table 4.

### Example 22

### Batch-Wise Alkylation Test with IL-8

Example 17 was repeated with using IL-8 instead of IL-3 The composition data of the alkylate product are given in table 5.

### Example 23

### Batch-Wise Alkylation Test with IL-9

Example 17 was repeated with using IL-9 instead of IL-3 The composition data of the alkylate product are given in table 5.

### Example 24

### Continuous Alkylation Test with IL-10

200 g of composite IL-10 was placed into a 500 mL autoclave. The autoclave was closed, the stirrer was started, and then the autoclave was controlled at 20° C. A C4 feed with an I/O ratio (isobutane/2-butene) of 20 mol/mol was stored in a feed storage tank. 1.0 kg of the C4 feed, after filtration, was pumped through a dryer, and then entered into the autoclave. The feed rate was controlled at 700 mL/h by the plunger pump. The pressure in the autoclave was maintained at 0.6 MPa to keep the reactants and product in liquid phase.

During reaction and filling the autoclave, the reaction system was separating into two phases due to the differences in density. The upper part of the reaction mixture in the autoclave was the unreacted feed and products, while the lower part consisted of a mixture of ionic liquid and hydrocarbons. When the autoclave was full and started to overflow a sample was taken under pressure from this overflow.

### Example 25

### Continuous Alkylation Test with IL-11

Example 24 was repeated with using IL-11 instead of IL-10. The composition data of the alkylate product are given in table 6.

### Example 26

### Continuous Alkylation Test with IL-12

Example 24 was repeated with using IL-12 instead of IL-10. The composition data of the alkylate product are given in table 6.

### Example 27

### Continuous Alkylation Test with IL-13 (Comparative Example)

Example 24 was repeated with using IL-13 instead of IL-10. The composition data of the alkylate product are given in table 6.

### Example 28

### Continuous Alkylation Test with IL-14 (Comparative Example)

Example 24 was repeated with using IL-14 instead of IL-10. The composition data of the alkylate product are given in table 6.

### Analysis of Feed and Products of Examples 15-28

### Hydrocarbon Composition of Feed:

The C4 feed (gas sample) was analyzed by an Agilent refinery gas analyzer (an Agilent 6890 gas chromatograph with Chem Station software) to determine the volume percentage of the components. Data were converted to mass percentages with the state equation of ideal gases. The water content of the C4 feed was measured by Karl-Fisher analyzer.

### Hydrocarbon Composition of Alkylate Product:

The alkylate products were analyzed by a GC SP3420, equipped with a flame ionization detector (FID). The components in the product were separated by a 50 m PONA capillary column (ID 0.25 mm, 0.25 μm film thickness). The temperatures of injector and detector were 250° C. and 300° C., respectively. The temperature program was as follows, holding at 40° C. for two minutes, increasing to 60° C. at a speed of 2° C./min, increasing to 120° C. at a speed of 1° C./min, increasing to 180° C. at a speed of 2° C./min, and finally holding at 180° C. for thirteen minutes. The hydrocarbons were identified by their retention time and quantitative analysis was done by their normalised areas.

The RON of alkylate was calculated according to the equation (1).

$$RON = {\sum }_{i = 1}^{n} {C}_{i} \cdot {RON}_{i} \qquad ( 1 )$$

In this equation, i is a component in alkylate, Cᵢ is the relative content of component i in alkylate, wt %, RONᵢ is the RON of component i.

### Chloride Content in Alkylate Product:

The total chlorine content in alkylate was measured by microcoulometer, and the chloride types and contents were measured by GC-ECD.

### Solids Content in IL:

Solid content of the ionic liquid layer was determined by centrifugation (with various temperature, time, and rotation speed). Thus “solid” means concentrated solid, and the precipitated paste still contain ILs.

Results from Alkylation Tests

TABLE 1
Alkylate composition.
Average of hydrocarbon samples taken from examples 15 and 16

| Alkylation | | C5-C7 | TMP | DMH | TMP/ | C9 | C9+ | | Cl |
|---|---|---|---|---|---|---|---|---|---|
| example | Catalyst | wt % | wt % | wt % | DMH* | wt % | wt % | RON | mg/L |
| 15 | IL-1 (Et₃NHCl) | 11.3 | 59.1 | 9.6 | 6.1 | 4.3 | 15.6 | 89.9 | 739 |
| 16 | IL-2 (BmimCl) | 9.7 | 67.7 | 10.9 | 6.1 | 2.2 | 9.3 | 91.7 | 282 |

*TMP = trimethylpentane; DMP = dimethylpentane

The results in Table 1 show that selectivity and RON of alkylate produced by catalysis with BmimCl composite ionic liquid (IL-2) are higher than alkylate produced using Et₃NHCl composite ionic liquid (IL-1).

TABLE 2
Catalyst lifetime of composite IL-1 and IL-2

| alkylation test & | | |
|---|---|---|
| Catalyst | C4 Feed (kg) | Olefin conversion (mol %) |
| Example 15 | 4.0 | 100.0 |
| IL-1 (Et₃NHCl based) | 5.5 | 14.6 |
| Example 16 | 7.0 | 100.0 |
| IL-2 (BmimCl based) | 9.3 | 15.0 |

The results in Table 2 show that the lifetime of BmimCl composite ionic liquid (IL-2) is longer than that of Et₃NHCl composite ionic liquid (IL-1).

TABLE 3
Solids formation of alkylation catalyzed by IL-1 and IL-2

| | | | Alkylate | Paste per |
|---|---|---|---|---|
| Alkylation | | Paste | produced* | alkylate |
| example | Catalyst | amount g | kg | g/kg |
| 15 | IL-1 (Et₃NHCl based) | 13.3 | 0.4 | 31.8 |
| 16 | IL-2 (BmimCl based) | 10.4 | 0.7 | 14.1 |

*Amount of alkylate produced until conversion dropped <100% conversion

The results in Table 3 show that the solids generation during alkylation catalyzed by BmimCl composite ionic liquid (IL-2) is less than with Et₃NHCl composite ionic liquid (IL-1).

TABLE 4
Composition of alkylate catalysed by
[Bmim]Cl—xAlCl₃—0.5CuCl

| Alkylation | x | C₅-C₇ | C₈ | C₉-C₉₊ | Cl | |
|---|---|---|---|---|---|---|
| example | mol/mol | wt % | wt % | wt % | mg/L | Conversion |
| 17 | 1.2 | 14.6 | 16.8 | 68.9 | 415 | 55 |
| 18 | 1.4 | 11.2 | 45.2 | 43.6 | 377 | 82 |
| 19 | 1.6 | 11.8 | 52.2 | 34.5 | 301 | 90 |
| 20 | 1.8 | 16.7 | 57.6 | 25.7 | 251 | 99 |
| 21 | 2.0 | 18.5 | 56.8 | 25.5 | 290 | 99 |

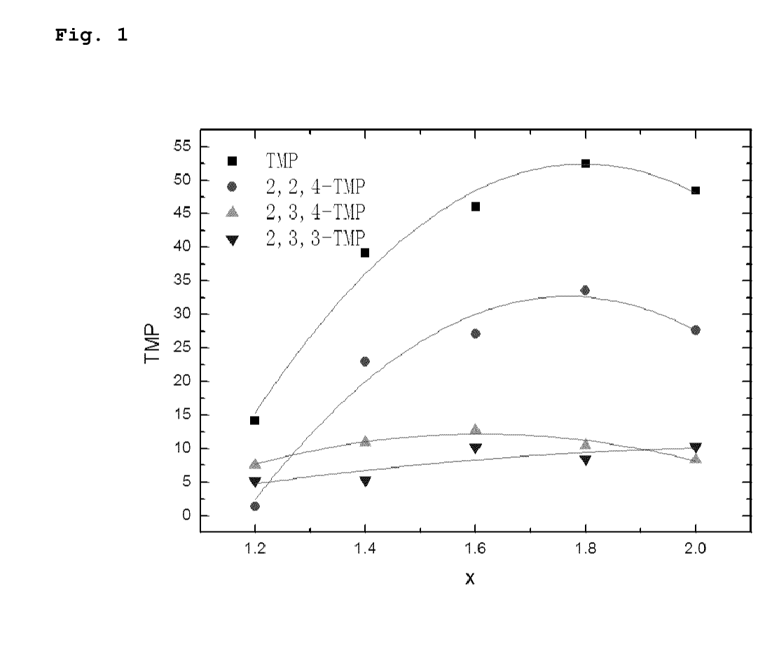

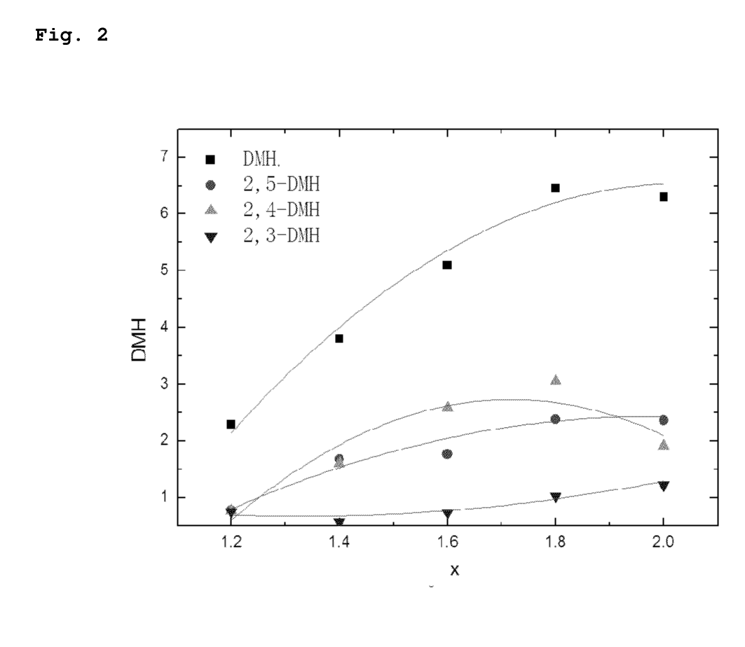

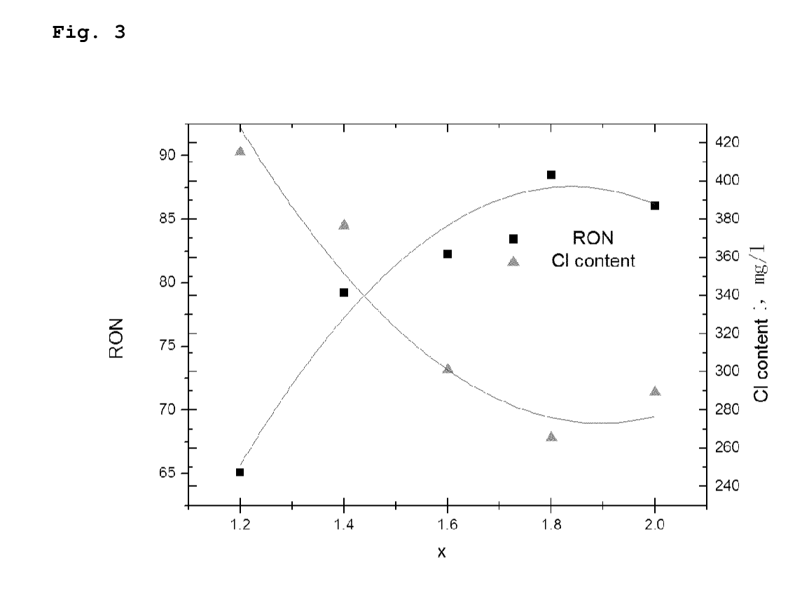

The results in Table 4 are graphically represented in FIGS. 1, 2 and 3. The results in Table 4 and FIG. 1, 2 and show that alkylation catalysed with [Bmim]Cl-xAlCl₃—CuCl shows the best performance, e.g. highest TMP and C8 selectivity, highest RON of alkylate and lowest Cl content, for x=1.8.

TABLE 5
Effect of different cations on C4 alkylation

| Alkylation | | C₅-C₇ | C₈ | C₉₊ | | Cl |
|---|---|---|---|---|---|---|
| example | Ionic liquid | w % | w % | w % | RON | mg/L |
| 20 | [Bmim]Cl—1.8AlCl₃—0.5CuCl | 16.7 | 57.6 | 25.7 | 88.9 | 251 |
| 22 | [t-Bmim]Cl—1.8AlCl₃—0.5CuCl | 12.0 | 64.4 | 23.5 | 90.4 | 41 |
| 23 | [i-Bmim]Cl—1.8AlCl₃—0.5CuCl | 15.3 | 59.3 | 25.3 | 89.7 | 280 |

The results of Table 5 show that [t-Bmim]C1-1.8AlCl₃-0.5CuCl composite ionic liquid gives the highest selectivity for C₈ production.

TABLE 6
Performance of alkylate catalyzed by IL-10 to IL-14

| | | catalyst preparation conditions | | Alkylate composition | | | | | |
|---|---|---|---|---|---|---|---|---|---|
| Alkylation | | Heating time after | Temp. | C₅-C₇ | TMP | DMH | TMP/ | C9⁺ | |
| example | | last AlCl₃ addition h | ° C. | wt % | wt % | wt % | DMH* | wt % | RON |
| 24 | IL-10 | 4 | 160 | 8.1 | 82.0 | 6.8 | 12.1 | 3.1 | 97.3 |
| 25 | IL-11 | 4 | 120 | 10.6 | 75.9 | 8.7 | 8.7 | 4.8 | 95.1 |
| 26 | IL-12 | 8 | 120 | 9.3 | 78.5 | 7.8 | 10.1 | 4.4 | 96.4 |
| 27 | IL-13 | 4 | 100 | 11.8 | 67.2 | 11.4 | 5.9 | 9.6 | 93.5 |
| 28 | IL-14 | 8 | 100 | 12.2 | 69.8 | 11.0 | 6.3 | 7.0 | 94.8 |

*TMP = trimethylpentane; DMP = dimethylpentane

The catalyst performance results shown in Table 6 show that both the selectivity for TMP and the RON of the alkylate were better when IL was used that was produced at higher temperatures than 100° C.

The results further show that prolonged mixing and heating of the synthesis mixture also leads to better catalyst performance (see IL-12 and IL-14, in comparison to IL-11 and IL-13, respectively).

In U.S. Pat. No. 7,285,698 the specific temperature range disclosed for synthesis of a composite ionic liquid was selected from 80° C. to 100° C. It has now been found that the synthesis temperature can be favourably selected at a higher range, e.g. at 120-170° C., resulting in composite ionic liquids with better alkylation performance.

1. A composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the ammonium cation is a N,N′-disubstituted imidazolium cation, optionally further substituted at the 2-, 4- and/or 5-positions, the substituents independently being selected from C1-C10 alkyl, and C6-C10 aryl.

2. The composite ionic liquid of claim 1, wherein the further metal is selected from copper, iron, zinc, nickel, cobalt, molybdenum, silver and platinum.

3. The composite ionic liquid of claim 1, wherein the aluminium salt is aluminium (III) chloride.

4. The composite ionic liquid of claim 1, wherein the composite coordinate anion comprises a copper salt as a further metal salt, preferably copper (I) chloride.

5. The composite ionic liquid of claim 1, wherein the substituents of the N,N′-disubstituted imidazolium cation are selected from C1-C6 alkyl and phenyl.

6. The composite ionic liquid of claim 1, wherein the cation is N-butyl, N′-methylimidazolium, preferably N-t-butyl, N′-methylimidazolium, optionally substituted with methyl at the 2-position.

7. The composite ionic liquid of claim 1, wherein the molar ratio of the aluminium salt to the ammonium salt ranges from 1.2 to 2.2, preferably 1.6 to 2.0, and more preferred 1.7 to 1.9, and most preferably the ratio is 1.8.

8. The composite ionic liquid of claim 1, wherein the molar ratio of the copper salt to the ammonium salt ranges from 0.3 to 0.7, preferably 0.4 to 0.6, most preferably the ratio is 0.5.

9. A process for preparing an alkylate comprising contacting in a reaction zone a hydrocarbon mixture comprising at least an isoparaffin and an olefin with a composite ionic liquid according to any one of the preceding claims.

10. A process for the preparation of a composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the two or more metal salts are mixed with the ammonium cations, in the form of an ammonium salt, and the mixture is kept at a temperature of 120 to 170° C. while stirring until all solids have completely converted into the liquid phase.

11. The process of claim 10, wherein the reaction mixture is kept at 120 to 160° C. for an extended period of time, preferably at least 4 hours, more preferred for at least 8 hours, up to about 12 hours, after the addition of all of the aluminium salt, preferably aluminium chloride.

12. The process of claim 10, wherein the composite ionic liquid is a composite ionic liquid comprising ammonium cations and composite coordinate anions derived from two or more metal salts, wherein at least one metal salt is an aluminium salt and any further metal salt is a salt of a metal selected from the group consisting of Group IB elements of the Periodic Table, Group IIB elements of the Periodic Table and transition elements of the Periodic Table, wherein the ammonium cation is a N,N′-disubstituted imidazolium cation, the substituents independently being selected from C1-C10 alkyl, and C6-C10 aryl.
